(19) United States
(12) Patent Application Publication
(10) Pub. No.: US 2024/0237893 A1
(43) Pub. Date: Jul. 18, 2024

(54) TEAR OSMOLARITY MEASUREMENT DEVICES AND ASSOCIATED METHODS

(71) Applicant: Paul Gavaris, Bethesda, MD (US)

(72) Inventor: Paul Gavaris, Bethesda, MD (US)

(21) Appl. No.: 18/415,158

(22) Filed: Jan. 17, 2024

Related U.S. Application Data
(60) Provisional application No. 63/439,472, filed on Jan. 17, 2023.

(51) Int. Cl. A61B 3/10; A61B 5/00; A61B 5/145; A61B 5/1468

(52) U.S. Cl. CPC A61B 3/101; A61B 5/0004; A61B 5/14507; A61B 5/1468; A61B 5/6821; A61B 2560/0214

(57) ABSTRACT

In one embodiment, a method for determination of tear osmolarity includes inserting a measurement device comprising a crescent-shaped body within an inferior fornix of a subject between an eyeball and an eyelid of the subject; contacting a sensor coupled to the crescent-shaped body with tear fluid of the subject; generating a measurement signal with the sensor indicative of a characteristic of the tear fluid; transmitting via a wireless communication module of the measurement device the measurement signal to a computing device; and processing with the computing device the measurement signal to generate a final measurement of the characteristic of the tear fluid in response to repeated stable measurements received from the measurement device.

### CROSS-REFERENCE TO RELATED APPLICATIONS

The present application claims the benefit of U.S. Provisional Application No. 63/439,472, entitled “TEAR OSMOLARITY MEASUREMENT DEVICE AND ASSOCIATED METHOD,” filed Jan. 17, 2023, the entirety of which is hereby incorporated by reference.

### TECHNICAL FIELD

The present specification generally relates to tear measurement devices and associated methods and, specifically, in vivo measurement of tear constituent concentrations, particularly osmolarity.

### BACKGROUND

Devices for in vivo measurement of tear constituent concentrations are often imprecise and/or inaccurate. For example, osmometers repeatedly fail to accurately measure osmolarity. That is, the same subject eyes may exhibit a range of different osmolarities, for instance, when measured at different but not widely spaced time intervals, eye to eye, the next day or by different technicians. Thus the devices as utilized today are clinically unreliable, yielding results with too large a standard deviation or measurement error. Moreover, existing devices may cause reflex tearing (RT) which may further cause inaccurate readings.

Accordingly, a need exists for alternative tear characteristic measurement devices and methods that provide for more precise and accurate measurements.

### SUMMARY

In one embodiment, a method for determination of tear osmolarity includes inserting a measurement device comprising a crescent-shaped body into inferior fornix of a subject between an eyeball and an eyelid of the subject; contacting a sensor coupled to the crescent-shaped body with tear fluid of the subject; generating a measurement signal with the sensor indicative of a characteristic of the tear fluid; transmitting via a wireless communication module of the measurement device the measurement signal to a computing device; and processing with the computing device the measurement signal to generate a final measurement of the characteristic of the tear fluid in response to repeated stable measurements received from the measurement device

In another embodiment, a method for determination of tear osmolarity includes inserting a measurement device into a fornix of an eye of a subject; operating an impedance sensor of the measurement device to output an impedance signal indicative of impedance of tear fluid of the eye; wirelessly transmitting the impedance signal to an external computing device spaced from the subject; and after a time indicative of ocular adaptation and acclimation of the measurement device to the eye, calculating an osmolarity or concentration value based on the impedance signal and displaying the same via a display of the external computing device.

In yet another embodiment, a measurement device for determining a tear fluid characteristic includes a body shaped for insertion in an eye of a subject between an eyeball and an eyelid of the subject without extending over a pupil or onto a cornea of the eye; a sensor mounted to or embedded in the body and configured to measure concentration of a constituent of a tear fluid of the subject, the sensor including a sensing surface configured to be in contact with the tear fluid; and a wireless communication module mounted to or embedded in the body and communicatively coupled to the sensor so as to receive concentration data signals from the sensor.

In yet another embodiment, a method for determination of tear-fluid composition placing a measurement device in contact with tear fluid on an eye of a subject; operating the measurement device to obtain data measuring a concentration of a selected constituent of the tear fluid at intervals; automatically processing the data to compare different measurements of the concentration taken at different times to determine attainment of a stable or state of equilibrium of the concentration; and automatically communicating the stable or equilibrium value to the subject via a user interface device.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a measurement device for measuring a characteristic of a tear fluid of a subject, according to one or more embodiments shown and described herein;

FIG. 2 schematically depicts a cross-sectional view of the measurement device of FIG. 1 positioned on an eye of a subject, according to one or more embodiments shown and described herein; and

FIG. 3 schematically depicts a method for measuring a characteristic of a tear fluid of a subject using the measurement device of FIG. 1, according to one or more embodiments shown and described herein.

### DETAILED DESCRIPTION

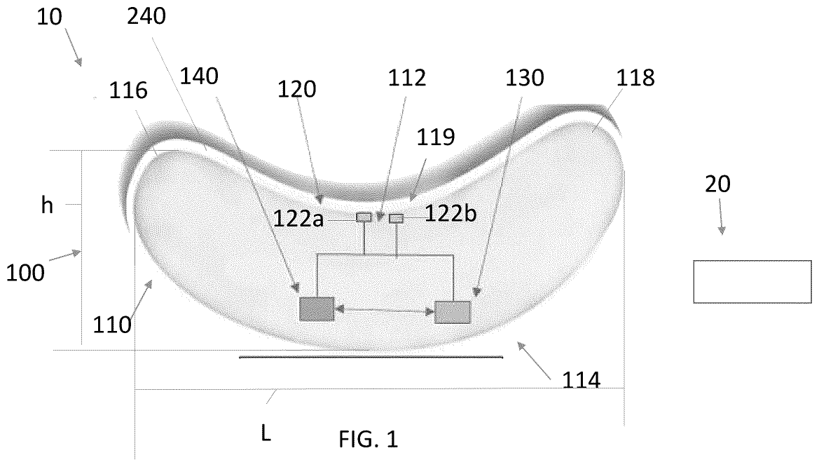

FIG. 1 generally depicts a measurement device for measuring a characteristic of a tear fluid of a subject, which may be removably positioned on a user's eye. Such characteristics may include but are not limited to biomarkers, protein, or other solutes. That is, a characteristic of a tear may relate to or include concentrations of specific solutes characteristically or uncharacteristically present in tear fluid/film. In particular, the measurement device may include a body, a sensor mounted to or embedded within the body, and a wireless communication module. As will be described in greater detail herein, the measurement device may be positioned on the eye of the user and the environment of the eye allowed to equilibrate or otherwise become accustomed to the device so as to provide accurate and consistent measurements, thereby providing improved diagnostic determinations for eye health.

As described above, existing devices for measuring tear fluid characteristics typically result in disruption of the environment of the eye leading to reflex tearing and incorrect instrument readings. In particular, traditional measuring techniques result in changes to the tear film (also referred to herein as tear fluid), its subtle dilution, change in blinking rate, the almost automatic welling of the tear meniscus, etc. In some cases, merely approaching the eye to perform the measurement may result in changes to the tear fluid volume such that measurements taken may not be accurate. Even light shone onto a subject's eye using a slit lamp biomicroscope may result in reflex tearing (RT). Unfortunately, these factors result in a large standard deviation in measurements of tear constituent or solutes materials.

Accurate measurements of tear fluid constituent components may be critical in determining eye health. In particularly, osmolarity is considered the single most important parameter for driving tear homeostasis. Accordingly, measuring the true osmolarity in real-time in-situ is highly desirable.

Embodiments of the present disclosure provide a measurement device which may be worn like a contact lens. It is noted that a device per the present disclosure need not use a whole contact lens as such could would affect the tear film osmolarity, increase adaptability time and potentially cause foreign body sensations leading to reflex tearing. Accordingly, in some embodiments the measurement device may include a crescent or banana-shaped body which may minimize disruption to the tear environment. For example, using a small banana-shaped contact lens segment, though large enough to contain any electronic components as described herein, eliminates or reduces such issues. Accordingly, tolerance adaptation and acclimation time may be minimized, without effect on vision and no foreign body sensations may be felt. Additional benefits may include reduced costs, increased margins, and higher acceptability and utility by patients and doctors alike.

Referring now to FIG. 1, a measurement device 100 for determining a characteristic of tear fluid according to one or more embodiments of the present disclosure is generally depicted. The measurement device 100 includes a body 110, a sensor 120, and a wireless communication module 130. In some embodiments, the measurement device 100 may further include a battery 140. A greater or fewer number of components may be included as part of the measurement device 100 without departing from the scope of the present disclosure. In some embodiments, the measurement device 100 may form part of a system 10 for determining a characteristic of tear fluid along with an external computing device 20. The system 10 may have any number of components without departing from the scope of the present disclosure.

The body 110 may be formed of any material which may be worn in contact with an eye of a user. In embodiments, the body 110 may be formed of a contact lens material. For example, the body 110 may be formed of a soft contact lens material such as hydrogel lens material, silicone hydrogen lens material, or the like. While it is contemplated that the body 110, may have a fully circular lens shape, in embodiments the body 110 may have a banana or crescent shape as depicted (such shape may also be described herein as an arcuate shape) such as when viewed from a front elevation as depicted in FIG. 1. Accordingly, the body 110 may have two opposing arcuate edges or sides 112, 114, each contiguous and attached to one another at apexes 116, 118. As depicted a first arcuate edge 112 may be concave and a second arcuate edge 114 may be convex. The convex edge or side may have a larger radius of curvature than the inner or concave edge or side.

The crescent shape may be particularly advantageous as such shape may be sized for insertion between the eyeball 200 and the eyelid 220 such as within or adjacent the inferior fornix 230. Such location may be desirable as it allows a subject to become acclimated to the position of the device with minimal disturbance.

As noted above, the body 110 may be sized and shaped to fit over the eyeball 200 such as between the eyeball 200 and the eyelid 220 (e.g., the lower eyelid 220). For example, the body 110 may provide the outer-most dimensions of the device. As illustrated in FIG. 1, the dimensions of the device may be about 10 mm or less length, L, by about 5 mm or less height, h, such as about 8 mm length, L, by about 5 mm, height, h. However, it is contemplated that a measurement device 100 as provided herein may have any dimensions and in some embodiments may be sized for the material anatomy of a subject to which it is to be applied. In embodiments, the size of the body 110 may be so unobtrusive to a subject that the subject becomes used to or insensitive to the measurement device 100, thereby reducing or eliminating reflex tearing.

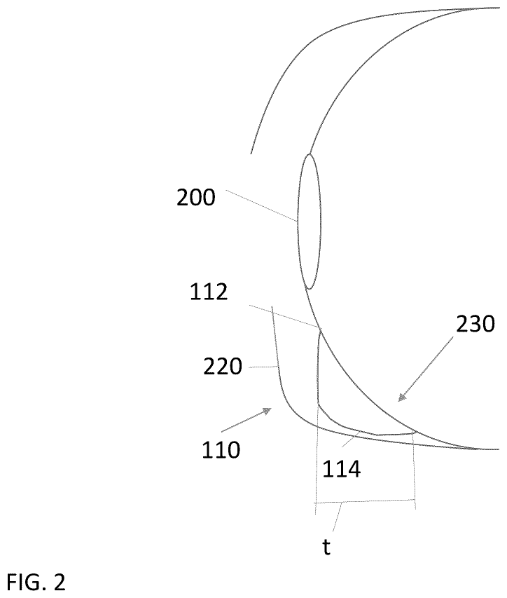

Referring now to FIG. 2, a schematic cross-section of the measurement device 100 is schematically depicted. As depicted, in some embodiments, the body 110 of the measurement device 100 may have a greater thickness, t, along or adjacent the second edge 114 of the body 110, such that the body 110 may be generally boomerang-shaped. Such increased thickness at the base may be useful for holding or containing functional hardware (e.g., battery 140, sensor 120, wireless communication module 130, related circuitry, or the like). In embodiments, the thickness, t, may be variable along a length of the device such that, for example, the thickness along the second edge 114 tapers to apexes 116, 118, depicted in FIG. 1. Such variable thickness may provide for smooth transitions to regions storing hardware and, thus, a more comfortable fit for the user/subject.

Still referring to FIG. 2, the measurement device 100 is illustrated as placed on an eye of the user. As noted above, the devices may be positioned between, e.g., laterally between, and in contact with the eyeball 200 and the eyelid 220 of the user, so as to be adjacent and/or within the inferior fornix 230. As depicted, the first edge 112 may be positioned facing upward away from the inferior fornix 230 which the second edge 114 is depicted facing downward toward the inferior fornix 230. As noted above and referring again to FIG. 1, the first edge 112 may be concave, accordingly, tear fluid 240 may sit within a region defined by the first edge 112. Accordingly, the first edge 112 may in embodiments provide a sensing surface 119 of the measurement device 100 described in greater detail below. As shown, the measurement device 100 may be sized so as to be positioned beneath the cornea and/or pupil and not extend over the cornea and/or pupil of the user. In embodiments, the measurement device 100 does not extend outside of the subject's lower eyelid 220.

As noted above, the measurement device 100 includes a sensor 120 for outputting a signal indicative of a characteristic of tear fluid of a user. The sensor 120 may include any number of sensors such as one or more sensors or a plurality of sensors. The sensor 120 may be any type suitable for detecting a characteristic of (e.g., solute in) tear fluid. In embodiments, the sensor 120 may be mounted to or embedded within the body 110 and configured to measure concentration of a constituent of tear fluid of a subject. In embodiments, the sensor 120 may be positioned on or within the sensing surface 119 and exposed to measure or output a signal indicative of the desired characteristic of the tear fluid. The sensor 120 may include any electrochemical sensor, such as an impedance sensor. Other electrochemical sensors may measure other electrical differentials set up between molecules within the tear fluid (e.g., potential, current, and/or conductivity). As an example illustration, in embodiments including an impedance sensor (which may include a current gauge and a voltage source, for example) the sensor 120 may include a plurality of electrodes 122a, 122b (e.g., sensing wires) exposed in the sensing surface 119 so as to be in operative or electrical contact with the tear fluid 240. The sensor 120 measures impedance created by the solutes in the tear fluid that is in contact with the sensing surface 119. Though two electrodes 122a, 122b are depicted, the sensor 120 may include a number of electrodes 122a. It is noted that while the electrodes 122a, 122b are illustrated as exposed at sensing surface 119, in embodiments, the electrodes 122a, 122b may be exposed along any surface of the body 110. Impedance measurements may be used to calculate tear osmolarity, for example.

Accordingly, in embodiments, the measurement device 100 may output a signal indicative of at least an osmolarity of the tear fluid in a subject's eye, such as impedance measurements which may be used to calculate osmolarity. However, the measurement device 100 may be provided with multiple electrode pairs having different solute-binding molecular elements or ligands to detect and measure the concentrations of a plurality of solutes in a tear sample, exemplarily including proteins and other complex molecules, bacteria, viruses, and bacterial antigens and viral antigens.

As noted above, the measurement device 100 includes a wireless communication module 130. The wireless communication module 130 may allow for wireless communication from the measurement device 100 to an external computing device 20. The wireless communication module 130 may be communicatively coupled to the sensor 120, such as wirelessly or via wired communication, and be able to transmit readings from the sensor 120 to the external computing device 20. For example, the wireless communication module 130 may include an antenna and/or other hardware for communicating via Wi-Fi, cellular, Bluetooth communication, or any communication hardware for communicating with the external computing device 20. For example, the wireless communication module 130 may include an antenna wire extending in the periphery of the body 110 along the opposing arcuate edges.

As will be described in greater detail, the wireless communication module 130 may receive readings from the sensor 120 and communicate such readings to the external computing device 20. In embodiments, such communication may be substantially constant or periodic. In some embodiments, a controller circuit or processor, may be programmed to cause the wireless communication module 130 to automatically and/or periodically communicate sensor readings such as between about every 0.5 seconds to about every 300 seconds. In some embodiments, such controller circuit may be integral within the measurement device 100 or control may be caused by the external computing device 20 in contact with the wireless communication module 130.

As noted hereinabove, the measurement device 100 may include a battery 140, which may be imbedded within the body 110. The battery 140 may be a single use or rechargeable battery. In some embodiments, the battery 140 may be inductively rechargeable. Being rechargeable may provide for increased study duration. The battery 140 may provide power to the sensor 120 and/or the wireless communication module 130.

As noted above, the measurement device 100 may communicate with an external computing device 20, such as via the wireless communication module 130. The external computing device 20 may be any computing device such as, but not limited to, a desktop computer, laptop computer, tablet, smart phone, or the like. The external computing device may link or communicate with the measurement device 100 to remotely operate and/or receive measurement signals from the measurement device 100. In embodiments, the external computing device may communicate with the measurement device 100 when within several feet or meters of the subject, however, greater distances are contemplated and possible. In some embodiments, the external computing device will communicate with the measurement device 100 via a network.

One or both of the measurement device 100 or the external computing device 20 may include a signal processor configured to compare different measurements of a concentration taken at different times to determine attainment of a stable or equilibrium state of the desired solute concentrations after insertion of the measurement device 100. For example, when the sensor 120 signal stays substantially constant for a predetermined period of time (e.g., 1 minute, 2 minutes, 5 minutes, 10 minutes, etc.), or for a predetermined number of measurements (10 measurements, 20 measurements, etc.), the stable or equilibrium state may be considered to have been met. For example, the processing of the impedance-measurement or -data signals may include comparing impedance or osmolarity values measured at different times after the inserting of the body 110 to ascertain whether a stable or equilibrium saline or other concentration state has been achieved. In embodiments, the external computing device 20 may be configured to automatically communicate the signals or equilibrium values to a user, e.g., via a display. The external computing device 20 may include a processor which executes non-transitory computer-readable instructions for calculating, from sensor signals, (and/or using electrochemical spectroscopy) a final or steady state numerical signals of the detected characteristic (e.g., osmolarity or the like). By waiting for a steady or equilibrium state changes in ion concentration such as caused by reflex tearing is avoided. Accordingly, accurate measurements may be taken.

As noted above, operating of the wireless communication module 130, which may be caused by the external computing device or via an internal control circuit, and may include transmitting the signals (e.g., impedance-measurement signals) with a predetermined periodicity, e.g., the periodicity of measurement. Alternatively, the successive measurements may be retained in a memory in the deployed measurement device 100 and transmitted in a burst as a bundle, for instance, upon approximation of the external device to the subject. In such embodiments, the measurement device 100 may include a memory for storying the data.

In embodiments, the periodicity may entail a regular time internal between about 0.5 second and about 300 seconds.

Accordingly, in view of the above, it is to be understood that tear characteristics may be measured with the measurement device 100 in situ.

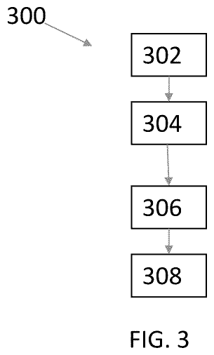

Referring now to FIG. 3, a method 300 of determining a characteristic of a tear fluid of a subject is generally depicted. A greater or fewer number of steps may be included without departing from the present disclosure. The method 300 may include at block 302 inserting the measurement device 100 in contact with tear fluid of a subject. For example the measurement device 100 may be inserted into or adjacent the inferior fornix 230 between the eyeball 200 and the eyelid 220 of the subject. At block 304 the method 300 may include contacting the sensor 120, such as described above, with the tear fluid. At block 306 the method 300 includes generating a measurement signal indicative of the characteristic of the tear fluid with the sensor 120. At block 308, the method 300 may include processing the signal to determine the characteristic. In some embodiments such determination may include processing the data to compare different measurements of the concentration taken at different times to determine attainment of a stable or equilibrium value of the concentration prior to determining the final measurement value.

In embodiments, the method 300 may include transmitting via the wireless communication module 130 the sensor 120 signal to the external computing device 20 for processing. For example, the external computing device 20 may process the measurement signal to generate a final measurement of the characteristic of the tear fluid. As noted above, the final measurement may be in response to repeated stable measurements received from the measurement device 100, as described above. For example, sensor 120 may periodically output an impedance or osmolarity signal, though other signals are contemplated and possible. In embodiments, processing the measurement signal may include comparing impedance or osmolarity values (for examples) measured at different times (e.g., such as at even time intervals between about 0.5 seconds and about 300 seconds) after the inserting the measurement device 100 into position on the user's eye. In embodiments, outputting of a final measurement may occur only after a time indicative of ocular adaptation and acclimation. For example, a standard amount of time (e.g., 1 minute, 5 minutes, 10 minutes, or at least 15 minutes) may be used. In other embodiments, the amount of time may be determined by comparing the impedance signal at different times to determine if the measurements are substantially consistent for a predetermined time period minutes (e.g., 1 minute, 5 minutes, 10 minutes, or at least 15 minutes) while the measurement device 100 is positioned adjacent or within the fornix. In some embodiments the external computing device may use the signal received to calculate osmolarity or other concentration values and display the same via a display of the external computing device.

It should now be understood that embodiments of the present disclosure contemplate the effective continuous monitoring of osmolarity or other tear fluid characteristics by measurement of tear electrolyte concentrations in situ. As described above, miniaturized electrolyte concentration measuring devices or sensors and a tiny wireless communication component may be integrated into a carrier or substrate body of, for example, a soft contact lens material. The sensor can be an impedance sensor that outputs an impedance measurement indicative of tear osmolarity based on electrolyte concentration. A series or stream of measurements from the sensor may be transmitted by the wireless communication module to an external computing device such as a smart phone or a computer that may display osmolarity data and/or stores the data for reference and further analysis.

As noted previously even very slight interactions of medical personnel (or anybody else) with a subject, particularly including the application of energy to the subject's eye or physical contact with the subject, can and does trigger reflex tearing that inevitably skews tests of tear composition, for instance, osmolarity. Accordingly, embodiments of the present disclosure provide for improved accuracy of measurements by allowing a subject to become acclimated to the measurement device for a sufficient time after placement of the measurement device. For example, it may be determined that the attainment of a stable or equilibration state is reached when the tear constituent concentrations, as transmitted by the measurement device, are found to have very similar numbers. In some embodiments, methods may further include a stable measurement for a predetermined time, prior to final determination or characteristic measurement. Such assures that the measured value is a trustworthy index of a subject's medical state or homeostasis.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method for determination of tear osmolarity, comprising:
inserting a measurement device comprising a crescent-shaped body within an inferior fornix of a subject between an eyeball and an eyelid of the subject;
contacting a sensor coupled to the crescent-shaped body with tear fluid of the subject;
generating a measurement signal with the sensor indicative of a characteristic of the tear fluid;
transmitting via a wireless communication module of the measurement device the measurement signal to a computing device; and
processing with the computing device the measurement signal to generate a final measurement of the characteristic of the tear fluid in response to repeated stable measurements received from the measurement device.

2. The method of claim 1, wherein the sensor is an electrochemical sensor comprising a plurality of electrodes configured to be in electronic communication with one another when in contact with the tear fluid.

3. The method of claim 1, wherein the sensor periodically outputs an impedance or osmolarity signal.

4. The method defined in claim 3, wherein processing the measurement signal comprises comparing impedance or osmolarity values measured at different times after the inserting the measurement device.

5. The method defined in claim 3, wherein the sensor periodically outputs the impedance or osmolarity signal at a regular time interval between about 0.5 second and about 300 seconds.

6. A method for determination of tear osmolarity, comprising:
inserting a measurement device within a fornix of an eye of a subject;
operating an impedance sensor of the measurement device to output an impedance signal indicative of impedance of tear fluid of the eye;
wirelessly transmitting the impedance signal to an external computing device spaced from the subject; and
after a time indicative of ocular adaptation and acclimation of the measurement device to the eye, calculating an osmolarity or concentration value based on the impedance signal and displaying the same via a display of the external computing device.

7. The method of claim 6, wherein the time indicative of ocular adaptation and acclimation of the measurement device comprises a time period as evidenced by readings of the impedance sensor being similar for at least 15 minutes while the measurement device is positioned within the fornix.

8. The method defined in claim 6, further comprising determining with the external computing device the time indicative of ocular adaptation and acclimation by comparing the impedance signal at different times to determine if the measurements are substantially consistent for a predetermined time period.

9. The method defined in claim 6, wherein the transmitting of the impedance signal comprises periodically transmitting the impedance signal at predetermined periodicity of between about 0.5 second and about 300 seconds.

10. The method defined in claim 6, wherein said measurement device has dimensions of about 8 mm by about 5 mm.

11. A measurement device for determining a tear fluid characteristic, the measurement device comprising:
a body shaped for insertion in an eye of a subject between an eyeball and an eyelid of the subject without extending over a pupil or onto a cornea of the eye;
a sensor mounted to or embedded in the body and configured to measure concentration of a constituent of a tear fluid of the subject, the sensor including a sensing surface configured to be in contact with the tear fluid; and
a wireless communication module mounted to or embedded in the body and communicatively coupled to the sensor so as to receive concentration data signals from the sensor.

12. The measurement device of claim 11, wherein said sensor is an electrochemical sensor that measures impedance of the tear fluid along the sensing surface.

13. The measurement device of claim 12, wherein the electrochemical sensor comprises plurality of electrodes exposed along the sensing surface.

14. The measurement device of claim 11, wherein the body has a crescent shape comprising two opposing arcuate edges each contiguous at a pair of apexes with the other of said two opposing arcuate edges.

15. The measurement device of claim 11, wherein the concentration is osmolarity of the tear fluid.

16. A system for determining a tear fluid characteristic, the system comprising:
the measurement device of claim 11; and
an external computing device in wireless communication with the measurement device, the external computing device comprising a signal processor configured to compare different measurements of the concentration taken at different times to thereby determine attainment of a stable or state of equilibrium value of the concentration, the external computing device being configured to automatically communicate said stable or equilibrium value to the subject.

17. A method for determination of tear-fluid composition, comprising:
placing a measurement device in contact with tear fluid on an eye of a subject;
operating the measurement device to obtain data measuring a concentration of a selected constituent of the tear fluid at intervals;
automatically processing the data to compare different measurements of the concentration taken at different times to determine attainment of a stable or state of equilibrium of the concentration; and
automatically communicating the stable or equilibrium value to the subject via a user interface device.

18. The method defined in claim 17, wherein the placing of the measurement device includes inserting the measurement device between an eyeball and an eyelid of the subject so that the measurement device does not extend over a cornea of the subject.

19. The method defined in claim 17, wherein the measurement device includes an inductively rechargeable battery.
